(19) United States
(12) Patent Application Publication
(10) Pub. No.: US 2020/0178772 A1
(43) Pub. Date: Jun. 11, 2020

(54) DISTAL END PORTION OF ENDOSCOPE, AND ENDOSCOPE HAVING THE SAME

(71) Applicant: OLYMPUS CORPORATION, Tokyo (JP)

(72) Inventor: Yuta SEKIGUCHI, Tokyo (JP)

(73) Assignee: OLYMPUS CORPORATION, Tokyo (JP)

(21) Appl. No.: 16/694,258

(22) Filed: Nov. 25, 2019

Related U.S. Application Data
(63) Continuation of application No. PCT/JP2018/015612, filed on Apr. 13, 2018.

(30) Foreign Application Priority Data
May 26, 2017 (JP) 2017-104689

(51) Int. Cl. A61B 1/00; A61B 1/307; F16H 25/14; G02B 23/24

(52) U.S. Cl. CPC A61B 1/00133; A61B 1/00128; G02B 23/2476; A61B 1/00183; F16H 25/14; A61B 1/307

(57) ABSTRACT

A distal end portion of an endoscope including a holding portion; a prism holding frame; a cam; a traction member that applies, to the prism holing frame, a first moment of causing the prism holding frame to rotate in one direction through traction; and an elastic member including a secured portion and a pressing portion, the elastic member being configured to generate a second moment in a direction opposite to a direction of the first moment in the prism holding frame using a force applied to the prism holding frame through the pressing of the pressing portion to cause the prism holding frame to rotate in another direction.

### CROSS REFERENCE TO RELATED APPLICATION

This application is a continuation application of PCT/JP2018/015612 filed on Apr. 13, 2018, and claims benefit of Japanese Application No. 2017-104689 filed in Japan on May 26, 2017, the entire contents of which are incorporated herein by this reference.

### BACKGROUND OF THE INVENTION

### 1. Field of the Invention

The present invention relates to a distal end portion of an endoscope that includes a first frame member and a second frame member to which an optical member is secured, the second frame member being turnably held by the first frame member, and to an endoscope that includes the distal end portion of the endoscope.

### 2. Description of the Related Art

In recent years endoscopes have widely been used in a medical field and an industrial field. Endoscopes used in the medical field enable observation of organs in body cavities and as needed, various treatments using treatment instruments inserted into insertion channels for the treatment instruments included in the endoscopes by inserting elongated insertion portions into body cavities of subjects.

Endoscopes used in the industrial field enable inspection such as observation of scratching, corrosion, and the like of sites to be examined in objects and various treatments and the like by inserting elongated insertion portions of the endoscopes into objects such as inside of jet engines and plant pipes.

Here, some endoscope used in the medical field include flexible insertion portions that are used for inspection and medical treatment of digestive tracts and the like while others include rigid insertion portions used for surgical operations.

In particular, examples of the endoscopes that include rigid insertion portions include rigid endoscopes, laparoscopes, nephroscopes, and the like.

In regard to endoscopes that include rigid insertion portions, a configuration in which a field of view (perspective angle) is freely changed by causing an objective lens or a prism provided at a distal end of an insertion portion to rotate in one direction or the other direction that is a direction opposite to the one direction as disclosed in Japanese Patent Application Laid-Open Publication No. 7-327916 and Japanese Patent No. 5932165, for example, is known.

Specifically, Japanese Patent Application Laid-Open Publication No. 7-327916 discloses a configuration in which a turning shaft of an objective lens that is an optical member is turnably fitted into a bearing hole of a holding portion that is a first frame member and a field of view is freely changed by causing the lens to rotate in one direction or the other direction using two wires.

Japanese Patent No. 5932165 discloses a configuration in which a turning shaft of a projecting portion that is a second frame member configured to hold a prism that is an optical member is turnably fitted into a bearing hole of a holding portion that is a first frame member, ends of an elastic member configured to cause a rotation force to be generated in a direction opposite to a direction of wire traction are respectively secured to the holding portion and the projecting portion, and a field of view is thus freely changed by causing the prism to rotate in one direction or the other direction using one wire and the elastic member.

### SUMMARY OF THE INVENTION

A distal end portion of an endoscope according to an aspect of the present invention includes: a first frame member at which a bearing is formed; a second frame member to which an optical member is secured, the second frame member including a turning shaft turnably held by the bearing; a cam disposed at the second frame member so as to be turnable around the turning shaft of the second frame member; a traction wire secured to the second frame member so as to apply, to the second frame member, a first moment of causing the second frame member to rotate in one direction through traction; and an elastic member including a secured portion that is secured to the first frame member and a pressing portion that abuts on the cam while pressing the cam, the elastic member being configured to generate a second moment in a direction opposite to the direction of the first moment in the second frame member using a force applied to the second frame member through the pressing of the pressing portion to cause the second frame member to rotate in another direction.

An endoscope including a distal end portion of an endoscope according to an aspect of the present invention includes: the distal end portion of the endoscope.

### BRIEF DESCRIPTION OF THE DRAWINGS

FIG. 1 is a perspective view schematically illustrating an endoscope that includes a distal end portion according to a first embodiment along with an external device and a monitor;

FIG. 2 is a partial perspective view schematically illustrating a field of view direction variable mechanism provided in a distal end portion of an insertion portion in FIG. 1;

FIG. 3 is a side view of the field of view direction variable mechanism in FIG. 2 when seen in a direction III in FIG. 2;

FIG. 4 is a side view illustrating a field of view direction variable mechanism provided in a distal end portion of an endoscope according to a second embodiment when a direction of a field of view is a front view direction;

FIG. 5 is a side view illustrating the field of view direction variable mechanism in FIG. 4 in a state in which the direction of the field of view has been deflected in an upward direction;

FIG. 6 is a side view illustrating the field of view direction variable mechanism in FIG. 4 in a state in which the direction of the field of view has been deflected in a downward direction;

FIG. 7 is a side view illustrating a field of view direction variable mechanism provided in a distal end portion of an endoscope according to a third embodiment when a direction of a field of view is a front view direction;

FIG. 8 is a side view illustrating the field of view direction variable mechanism in FIG. 7 in a state in which the direction of the field of view has been deflected in the upward direction;

FIG. 9 is a side view illustrating the field of view direction variable mechanism in FIG. 7 in a state in which the direction of the field of view has been deflected in the downward direction;

FIG. 10 is a side view illustrating a field of view direction variable mechanism provided in a distal end portion of an endoscope according to a fourth embodiment when a direction of a field of view is a front view direction;

FIG. 11 is a side view illustrating the field of view direction variable mechanism in FIG. 10 in a state in which the direction of the field of view has been deflected in the upward direction;

FIG. 12 is a side view illustrating the field of view direction variable mechanism in FIG. 10 in a state in which the direction of the field of view has been deflected in the downward direction;

FIG. 13 is a side view illustrating a field of view direction variable mechanism provided in a distal end portion of an endoscope according to a fifth embodiment when a direction of a field of view is a front view direction;

FIG. 14 is a side view illustrating a field of view direction variable mechanism provided in a distal end portion of an endoscope according to a sixth embodiment when a direction of a field of view is a front view direction;

FIG. 15 is a plan view illustrating a cam and a turning shaft in FIG. 13 and the cam in FIG. 14 when seen in a direction XV in FIGS. 13 and 14 in an aligned manner;

FIG. 16 is a side view illustrating a field of view direction variable mechanism provided in a distal end portion of an endoscope according to a seventh embodiment when a direction of a field of view is a front view direction; and

FIG. 17 is a front view of a prism and a cam in FIG. 16 when seen in a direction XVII in FIG. 16.

### DETAILED DESCRIPTION OF THE PREFERRED EMBODIMENTS

Hereinafter, embodiments of the present invention will be described with reference to drawings. Note that the drawings are schematic diagrams. Note also that relationships between thicknesses and widths of the respective members, ratios of the thicknesses of the respective members, and the like are different from actual relationships and ratios, and it is a matter of course that parts with mutually different dimensional relationships and ratios are included across the drawings.

### First Embodiment

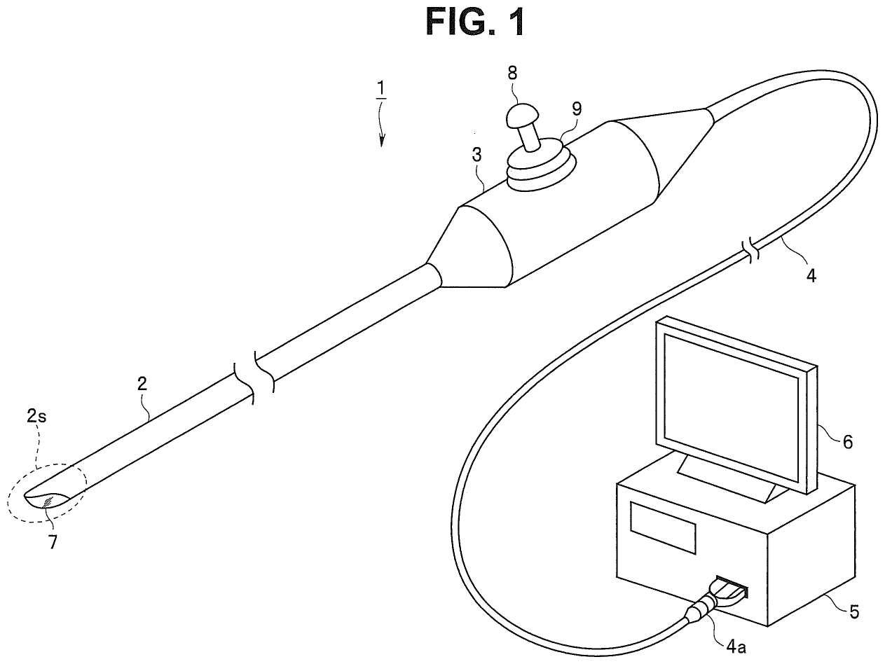

FIG. 1 is a perspective view schematically illustrating an endoscope that includes a distal end portion according to an embodiment along with an external device and a monitor.

As illustrated in FIG. 1, an endoscope 1 is adapted to be inserted into a subject for surgical use or to inspect and treat urinary organs, for example, includes a configuration for optically picking up images of a predetermined site to be observed in a subject.

Note that the subject into which the endoscope 1 is inserted is not limited to human bodies and may be other living bodies or artificial objects such as machines or buildings.

Main parts of the endoscope 1 are configured to include a rigid insertion portion 2 that is inserted into the subject, an operation portion 3 that is continuously provided at a proximal end of the insertion portion 2, a universal cord 4 extending from the operation portion 3, and a connector 4a provided at an extending end of the universal cord 4 and connected to an external device 5.

Note that as the endoscope 1 described as an example in the embodiment, a so-called rigid endoscope, a laparoscope, a nephroscope, or the like in which the insertion portion 2 does not include a flexible portion is exemplified.

It is a matter of course that the configuration according to the embodiment can also be applied to a flexible endoscope such as an upper endoscope to be inserted into an oral cavity and a lower endoscope to be inserted into an anus.

An image processing portion, which is not illustrated, is provided at the external device 5. The image processing portion generates a video signal on the basis of an image pickup device output signal outputted from an image pickup device that is provided at the endoscope 1, which is not illustrated, and outputs the video signal to a monitor 6.

In other words, an optical image (endoscope image) picked up by the image pickup device is displayed as a video on the monitor 6 in the embodiment.

Note that the image pickup device is a significantly small-scaled electronic component in which a plurality of devices configured to output electrical signals in accordance with incident light at a predetermined timing are aligned in a plane-shaped light receiving portion, and for example, schemes typically called a charge coupled device (CCD), a complementary metal-oxide semiconductor (CMOS) sensor, and the like or other various schemes are exemplified.

A dome-shaped cover glass 7 that serves as an observation window is provided at a distal end of the insertion portion 2 of the endoscope 1.

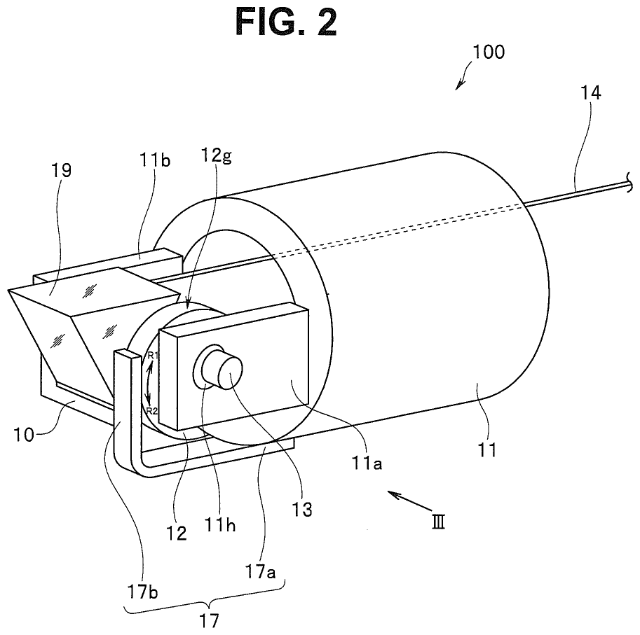

Note that a field of view direction variable mechanism 100 (see FIG. 2) is provided at a position that faces the cover glass 7 in the insertion portion 2, that is, in a distal end portion 2s of the insertion portion 2.

Further, an operation lever 8 that is an operation member of a so-called joy stick type to be operated to vary a direction of a field of view of the endoscope 1 is provided at the operation portion 3 of the endoscope 1, and a rubber boot 9 that covers a root portion of the operation lever 8 is provided at the root portion.

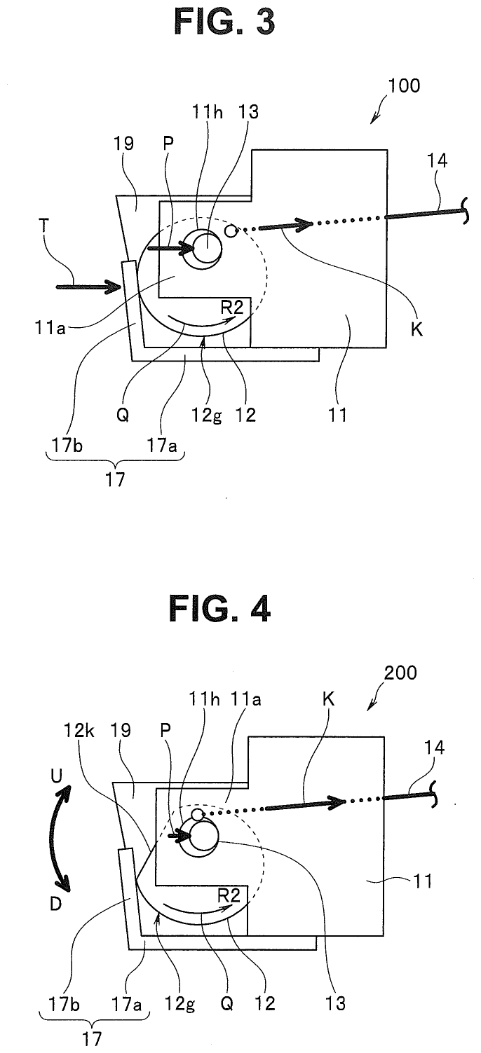

Next, the field of vie direction variable mechanism provided at the insertion portion 2 of the endoscope 1 will be described using FIGS. 2 and 3.

FIG. 2 is a partial perspective view schematically illustrating the field of view direction variable mechanism provided in the distal end portion of the insertion portion in FIG. 1, and FIG. 3 is a side view of the field of view direction variable mechanism in FIG. 2 when seen in a direction III in FIG. 2.

As illustrated in FIGS. 2 and 3, the field of view direction variable mechanism 100 includes a holding portion 11 that is a first frame member.

The holding portion 11 is formed into a substantially cylindrical shape using metal such as stainless steel or a hard resin, for example, and two arm portions 11a and 11b projecting forward in a longitudinal axis direction of the holding portion 11 are provided at a distal end so as to face each other.

Also, bearing holes 11h that are bearings with circular outer shapes are respectively formed in the respective arm portions 11a and 11b so as to penetrate in a direction that substantially perpendicularly intersects with the longitudinal axis direction.

A turning shaft 13 of a prism holding frame 10 that is a second frame member is turnably fitted into the bearing holes 11h.

The prism holding frame 10 has an L-shaped section, and a prism that is an optical member is secured to the prism holding frame 10 through adhesion, for example.

A cam 12 with a circular outer circumferential surface 12g is secured to a portion facing one side of the arm portion 11a of the prism holding frame 10.

Turning shafts 13 respectively provided at the cam 12 and the portion of the prism holding frame 10 that faces the arm portion 11b are respectively fitted into the respective bearing holes 11h of the arm portions 11a and 11b. In this manner, the prism holding frame 10 is held by the holding portion 11 so as to be turnable in one direction R1 or the other direction R2.

In this manner, the prism holding frame 10 is provided so as to be turnable around the turning shafts 13 in a state in which the prism holding frame 10 is sandwiched between the two arm portions 11a and 11b.

Note that as illustrated in FIGS. 2 and 3, the turning shaft 13 provided at the cam 12 is located with upward eccentricity from the center of the circular cam 12.

A distal end of one traction wire 14 that applies, to the prism holding frame 10, a first moment K that is a traction force of pulling the prism holding frame 10 through an operation of inclining the operation lever 8 and causing the prism holding frame 10 to rotate in the one direction R1 is secured to the prism holding frame 10 through soldering, swaging, or the like.

The traction wire 14 extends up to the operation portion 3 through the inside of the holding portion 11. Note that the proximal end of the traction wire 14 is connected to the operation lever 8 provided at the operation portion 3.

Further, a secured portion 17a of an L-shaped elastic member 17 is secured to the holding portion 11. Note that although a plate spring is exemplified as the elastic member 17 in the embodiment, a wire spring, a torsion spring, or the like may also be used.

A pressing portion 17b that is formed continuously with the secured portion 17a and abuts on the cam 12 while pressing the outer circumferential surface 12g of the cam 12 is provided at the elastic member 17.

The pressing portion 17b generates, in the prism holding frame 10, a second moment Q that is opposite to the first moment K and causes the prism holding frame 10 to rotate in the other direction R2 using an elastic force T applied from the pressing portion 17b to the cam 12 by pressing the cam 12.

As a result, a force that is opposite to the traction direction of the traction wire 14 is applied to the prism holding frame 10. Therefore, the traction wire 14 is constantly in a state in which a tensile force is applied by the second moment Q even in a non-traction state.

The pressing portion 17b constantly applies, to the turning shafts 13, a force P of pressing the turning shafts 13 against the bearing holes 11h in one direction by pressing the cam 12 with the elastic force T.

Note that other configurations of the field of view direction variable mechanism 100 are the same as configurations in the related art.

According to the field of view direction variable mechanism 100 configured as described above, while the traction wire 14 is pulled through the operation of inclining the operation lever 8, and the first moment K is applied to the prism holding frame 10 against the second moment Q applied from the pressing portion 17b to the prism holding frame 10, the prism holding frame 10 rotates in the one direction R1, that is, the upward direction in FIGS. 2 and 3.

In this manner, a light refraction direction of the prism 19 held by the prism holding frame 10 changes, and the direction of the field of view of the endoscope 1 is deflected upward from the front view direction based on the light receiving surface of the image pickup device.

On the other hand, if the operation of inclining the operation lever 8 ends, then the second moment Q is applied from the pressing portion 17b of the elastic member 17 to the prism holding frame 10, and the prism holding frame 10 thus rotates in the other direction R2, that is, the downward direction in FIGS. 2 and 3.

In this manner, the light refraction direction of the prism 19 held by the prism holding frame 10 changes, and the direction of the field of view of the endoscope 1 is deflected downward on the basis of the receiving surface of the image pickup device from the front view direction.

As described above, the embodiment in which the one traction wire 14 applies, to the prism holding frame 10, the force of causing the prism holding frame 10 to rotate in the one direction R1 by applying the first moment K to the prism holding frame 10 has been described.

Also, the embodiment in which the elastic member 17 applies, to the prism holding frame 10, the force of causing the prism holding frame 10 to rotate in the other direction R2 by the pressing portion 17b applying the elastic force T to the outer circumferential surface 12g of the cam 12 and applying the second moment Q to the prism holding frame 10 has been described.

Since this causes the turning shafts 13 to be constantly pressed against the bearing holes 11h in the one direction with the force P applied to the turning shafts 13 by the elastic force T, the turning shafts 13 do not cause backlash with respect to the bearing holes 11h. Therefore, it is possible to prevent degradation of images and abrupt changes in field of view due to deviation of an image pickup optical axis.

It is also possible to cause the prism holding frame 10 to rotate in the other direction R2 even if only one traction wire 14 is inserted into the insertion portion 2 by the second moment Q being applied from the pressing portion 17b to the prism holding frame 10.

Therefore, it is possible to cause the direction of the field of view of the endoscope 1 to vary in the two directions with the one traction wire 14. Accordingly, it is possible to realize reduction of a diameter and a scale of the insertion portion 2.

As described above, it is possible to provide the distal end portion 2s of the endoscope 1 and the endoscope 1 that includes the distal end portion 2s of the endoscope 1 with the configuration with which it is possible to prevent backlash of the turning shafts 13 of the prism 19 without leading to an increase in diameter of the insertion portion 2.

### Second Embodiment

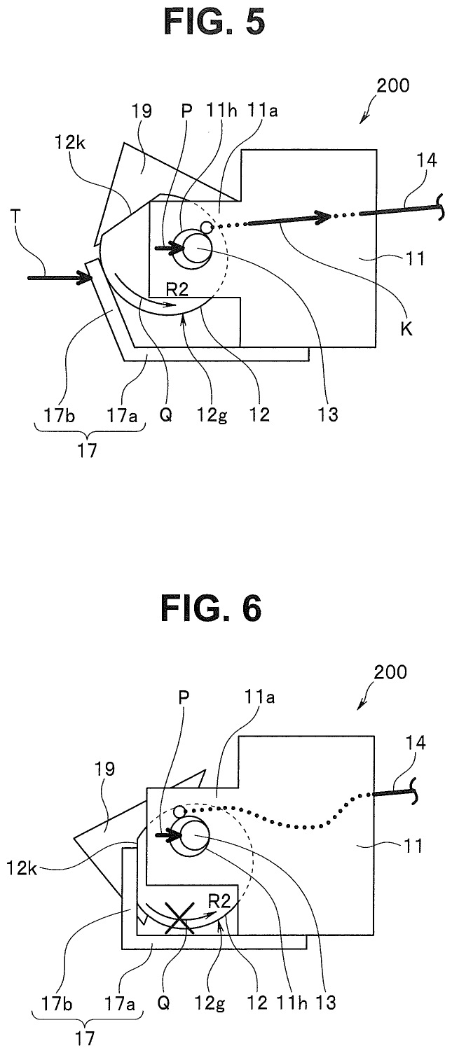

FIG. 4 is a side view illustrating a field of view direction variable mechanism provided in a distal end portion of an endoscope according to the present embodiment when a direction of the field of view is a front view direction, FIG. 5 is a side view illustrating the field of view direction variable mechanism in FIG. 4 in a state in which the direction of the field of view has been deflected in an upward direction, and FIG. 6 is a side view illustrating the field of view direction variable mechanism in FIG. 4 in a state in which the direction of the field of view has been deflected in a downward direction.

Configurations of a distal end portion of an endoscope and an endoscope that includes the distal end portion of the endoscope according to a second embodiment are different in that a notch is formed in an outer circumferential surface of a cam as compared with the distal end portion of the endoscope according to the aforementioned first embodiment illustrated in FIGS. 1 to 3.

Therefore, only the different point will be described, same reference numerals will be given to components that are similar to the components in the first embodiment, and description of the similar components will be omitted.

As illustrated in FIGS. 4 to 6, a notch 12k is formed in a plane shape at a portion of an outer circumferential surface 12g of a cam 12 in a field of view direction variable mechanism 200, with which a pressing portion 17b comes into contact.

The notch 12k has a function of causing the rotation of the cam 12 in the other direction R2 to stop by the pressing portion 17b abutting on the notch 12k when the second moment Q is applied from the pressing portion 7I to the prism holding frame 10 and the prism holding frame 10 rotates in the other direction R2.

Note that the other configurations are the same as the configurations in the aforementioned first embodiment.

According to such a configuration, there is a probability that the prism holding frame 10 rotates in the other direction R2 more than expected due to the second moment Q and the prism 19 comes into contact with another member in a case in which plastic elongation occurs in the traction wire 14 as illustrated in FIG. 6 due to repeated utilization.

Note that a configuration in which a stopper configured to secure a position of the traction wire is provided at the operation portion 3 such that a relaxation force that is opposite to the traction force does not act on the traction wire 14 in a state in which the traction wire 14 is pulled is also known in the related art.

However, since the configuration is a configuration in which the position of the traction wire 14 on the side of the proximal end is secured, it is not possible to stop, at a prescribed position, the rotation position of the prism holding frame 10 in the other direction R2 due to the application of the second moment Q in a case in which plastic elongation has occurred on the side of the distal end of the traction wire 14 as illustrated in FIG. 6.

However, according to the embodiment, it is possible to reliably stop the rotation in the other direction R2 at a prescribed position even if plastic elongation has occurred in the traction wire 14 as illustrated in FIG. 6 since the pressing portion 17b comes into contact with the notch 12k.

Note that the other advantages are same as the advantages of the aforementioned first embodiment.

### Third Embodiment

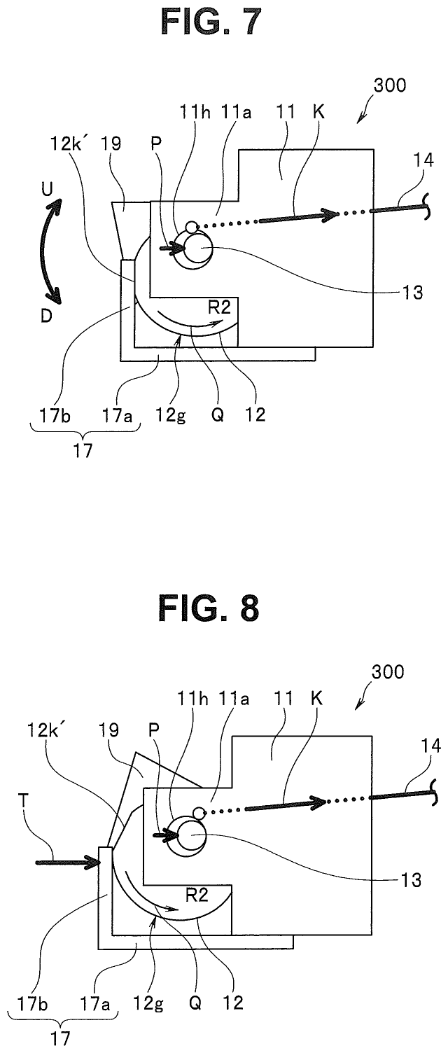

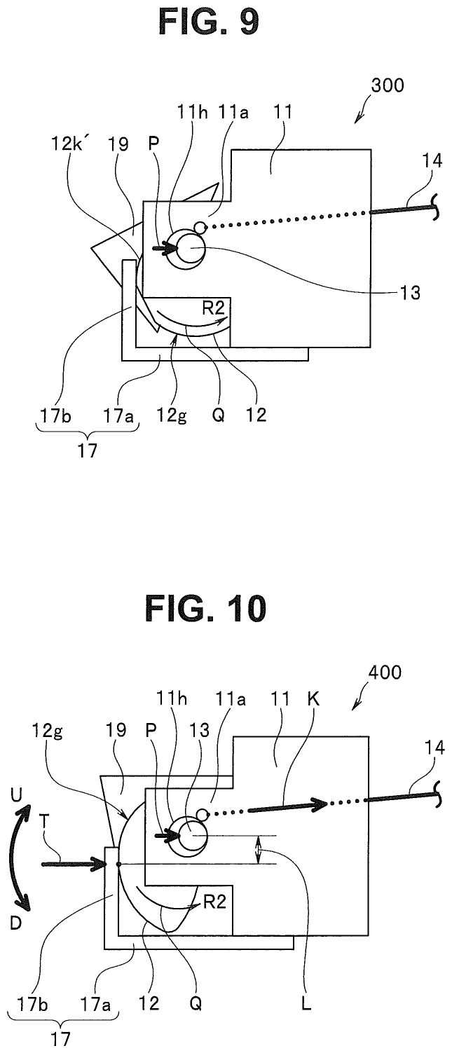

FIG. 7 is a side view illustrating a field of view direction variable mechanism provided in a distal end portion of an endoscope according to the present embodiment, when a direction of the field of view is a front view direction, FIG. 8 is a side view illustrating the field of view direction variable mechanism in FIG. 7 in a state in which the direction of the field of view has been deflected in the upward direction, and FIG. 9 is a side view illustrating the field of view direction variable mechanism in FIG. 7 in a state in which the direction of the field of view has been deflected in the downward direction.

A configuration of the distal end portion of the endoscope and the endoscope that includes the distal end portion of the endoscope according to a third embodiment is different in the size of the notch formed in the outer circumferential surface of the cam as compared with the distal end portion of the endoscope according to the aforementioned second embodiment illustrated in FIGS. 4 to 6.

Therefore, only the different point will be described, same reference numerals will be given to components that are similar to the components in the second embodiment, and description of the components will be omitted.

As illustrated in FIGS. 7 to 9, a notch 12k′ is formed in a plane shape at a portion of an outer circumferential surface 12g of a cam 12 in a field of view direction variable mechanism 300, with which the pressing portion 17b comes into contact, in the field of view in the front view direction as illustrated in FIG. 7. Note that the notch 12k′ is formed to be smaller than the notch 12k illustrate in FIGS. 4 to 6.

The notch 12k′ has a function of allowing an operator to recognize the front view direction without seeing the operation lever 8 by the pressing portion 17b abutting on the notch 12k′ and causing vibration in the traction wire 14 to generate a click sensing when the first moment K is applied to the prism holding frame 10 by the traction wire 14 and an the prism holding frame 10 rotates in the one direction R1 such that the direction of the field of view is changed from the downward direction to the front view direction as illustrated in FIGS. 9 and 7 or when the second moment Q is applied from the pressing portion 17b to the prism holding frame 10 and the prism holding frame 10 rotates in the other direction R2 such that the direction of the field of view is changed from the upward direction to the front view direction as illustrated in FIGS. 8 and 7.

Therefore, a configuration that can cause a click sensing is not limited to the notch 12k′, and a projection, a recessed portion, or the like may be formed in the outer circumferential surface 12g. The generation of the click sensing using the notch 12k′ is not limited to the field of view in the front view direction.

Further, since the notch 12k′ according to the embodiment is smaller than the notch 12k according to the second embodiment, the notch 12k′ does not have a function of causing the rotation of the cam 12 in the other direction R2 to stop as in the second embodiment.

Note that the other advantages are same as the advantages of the aforementioned second embodiment.

### Fourth Embodiment

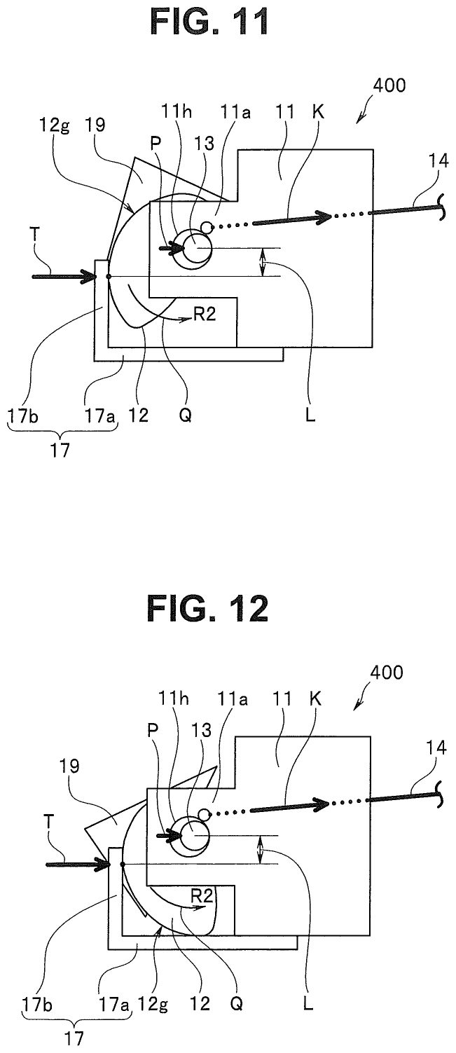

FIG. 10 is a side view illustrating a field of view direction variable mechanism provided in a distal end portion of an endoscope according to the present embodiment, when a direction of the field of view is a front view direction, FIG. 11 is a side view illustrating the field of view direction variable mechanism in FIG. 10 in a state in which the direction of the field of view has been deflected in the upward direction, and FIG. 12 is a side view illustrating the field of view direction variable mechanism in FIG. 10 in a state in which the direction of the field of view has been deflected in the downward direction.

Configurations of a distal end portion of an endoscope and an endoscope that includes the distal end portion of the endoscope according to a fourth embodiment are different in that an outer circumferential surface of a cam is formed to follow an involute curve as compared with the distal end portion of the endoscope according to the aforementioned first embodiment illustrated in FIGS. 1 to 3.

Therefore, only the different point will be described, same reference numerals will be given to components that are similar to the components in the first embodiment, and description of the components will be omitted.

As illustrated in FIGS. 10 to 12, an outer circumferential surface 12g of a cam 12 in a field of view direction variable mechanism 400 is formed to follow a known involute curve based on the turning shafts 13.

Note that the other configurations are same as the configurations in the aforementioned first embodiment.

According to such a configuration, a distance between the pressing portion 17b and the center of the turning shafts 13 with respect to the outer circumferential surface 12g slightly deviates with turning of the prism holding frame 10 among the field of view in the upward direction, the field of view in the front view direction, and the field of view in the downward direction. Therefore, there is a probability that the amount and the direction of the force P with which the turning shafts 13 are pressed against the bearing holes 11h slightly deviate due to the turning position.

However, since a distance L between the pressing portion 17b and the center of the turning shafts 13 with respect to the outer circumferential surface 12g is always constant regardless of the turning of the prism holding frame 10 as illustrated in FIGS. 10 to 12, the amount and the direction of the force P with which the turning shafts 13 are pressed against the bearing holes 11h are always constant in the embodiment. Therefore, it is possible to further curb backlash as compared with the first embodiment.

Note that the other advantages are same as the advantages of the aforementioned first embodiment.

### Fifth Embodiment

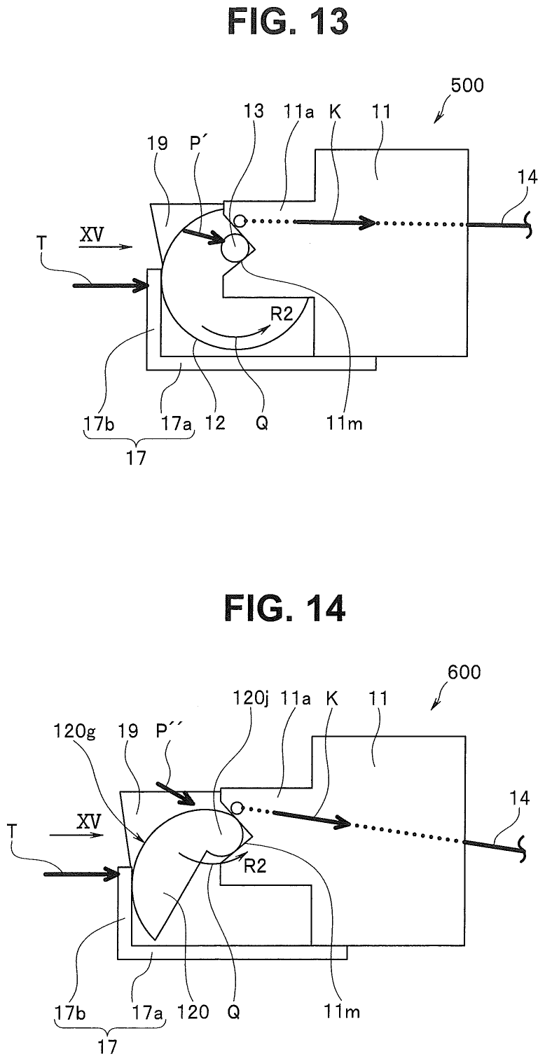

FIG. 13 is a side view illustrating a field of view direction variable mechanism provided in a distal end portion of an endoscope according to the present embodiment when a direction of a field of view is a front view direction.

Configurations of a distal end portion of an endoscope and an endoscope that includes the distal end portion of the endoscope according to a fifth embodiment are different in that bearing holes are formed into a V shape as compared with the distal end portion of the endoscope according to the aforementioned first embodiment illustrated in FIGS. 1 to 3.

Therefore, only the different point will be described, same reference numerals will be given to components that are similar to components in the first embodiment, and description of the components will be omitted.

As illustrated in FIG. 13, bearing holes 11m are formed into a V shape, for example, that is opened forward in the longitudinal axis direction of the holding portion 11 at the respective arm portions 11a and 11b in a field of view direction variable mechanism 500 according to the embodiment.

The turning shafts 13 are turnably fitted into the bearing holes 11m. Note that the shape of the bearing holes 11 are not limited to the V shape, and the bearing holes 11 may be formed into a recessed shape other than the V shape, for example, a semicircular shape or a U shape.

Note that the other configurations are same as the configurations in the aforementioned first embodiment.

According to such a configuration, backlash is less likely to occur than in the circular bearing holes 11h, and it is easy to perform assembly since the turning shafts 13 are pressed against two planes of the bearing holes 11m with a force P with respect to the bearing holes 11m due to the elastic force T applied to the cam 12 by the pressing portion 17b.

Note that the other advantages as same as the advantages of the aforementioned first embodiment.

### Sixth Embodiment

FIG. 14 is a side view illustrating a field of view direction variable mechanism provided in a distal end portion of an endoscope according to the present embodiment when a direction of a field of view is a front view direction, and FIG. 15 is a plan view illustrating the cam and the turning shaft in FIG. 13 and the cam in FIG. 14 when seen in a direction XV in FIGS. 13 and 14 in an aligned manner.

Configurations of a distal end portion of an endoscope and an endoscope that includes the distal end portion of the endoscope according to a sixth embodiment are different in that a cam and a turning shaft are integrally formed as compared with the distal end portion of the endoscope according to the aforementioned fifth embodiment illustrated in FIG. 13.

Therefore, only the different point will be described, same reference numerals will be given to components that are similar to the components in the fifth embodiment, and description of the components will be omitted.

As illustrated in FIG. 14, a cam 120 and a turning shaft 120j are integrally formed in a field of view direction variable mechanism 600 according to the embodiment.

Specifically, the turning shaft 120j that has a thickness that is substantially equal to a thickness V2 of the cam 120 and that is located so as to overlap with the thickness V2 in plan view from the side further forward than the prism 19 is formed at a part of the cam 120.

In other words, an abutting position of the pressing portion 17b with respect to the outer circumferential surface 120g and a fitting position of the turning shaft 120j in the bearing hole 11m are located in a same plane shape in plan view of the cam 120 from the side further forward than the prism 19.

The turning shaft 120j is turnably fitted into the V-shaped bearing hole 11m.

The turning shaft 120j is pressed against two planes of the bearing holes 11m with respect to the bearing hole 11m with a force P″ due to the elastic force T applied to the cam 120 by the pressing portion 17b. This prevents backlash of the turning shaft 120j with respect to the bearing hole 11m.

In addition, a rotation force in the one direction R1 is applied to the prism holding frame 10 by the first moment K applied to the prism holding frame 10 by the traction wire 14, the second moment Q is further applied to the cam 120 by the elastic force T applied to the cam 120 by the pressing portion 17b, and a rotation force in the other direction R2 is applied to the prism holding frame 10.

Note that in the embodiment, only a portion of the turning shaft 120j that is inserted into the bearing hole 11m has an outer circumference formed into a circular shape.

Note that the other configurations are same as the configurations in the aforementioned fifth embodiment.

According to such a configuration, on the assumption that the thickness of the cam 12 and the turning shaft 13 in FIG. 13 is defined as V1 and the thickness of the cam 120 is defined as V2 as illustrated in FIG. 15, the thickness in the embodiment is thinner (V2<V1) in the case in which the turning shaft 120j is integrally provided with the cam 120 as illustrated in FIG. 14 than in the case in which the turning shaft 13 is provided separately from the cam 12 as illustrated in FIG. 13.

Therefore, it is possible to further reduce the scale of the field of view direction variable mechanism 600, that is, it is possible to realize reduction of a scale and a diameter of the distal end portion 2s in which the field of view direction variable mechanism 600 is provided.

Note that other advantages are same as the advantages of the aforementioned fifth embodiment.

### Seventh Embodiment

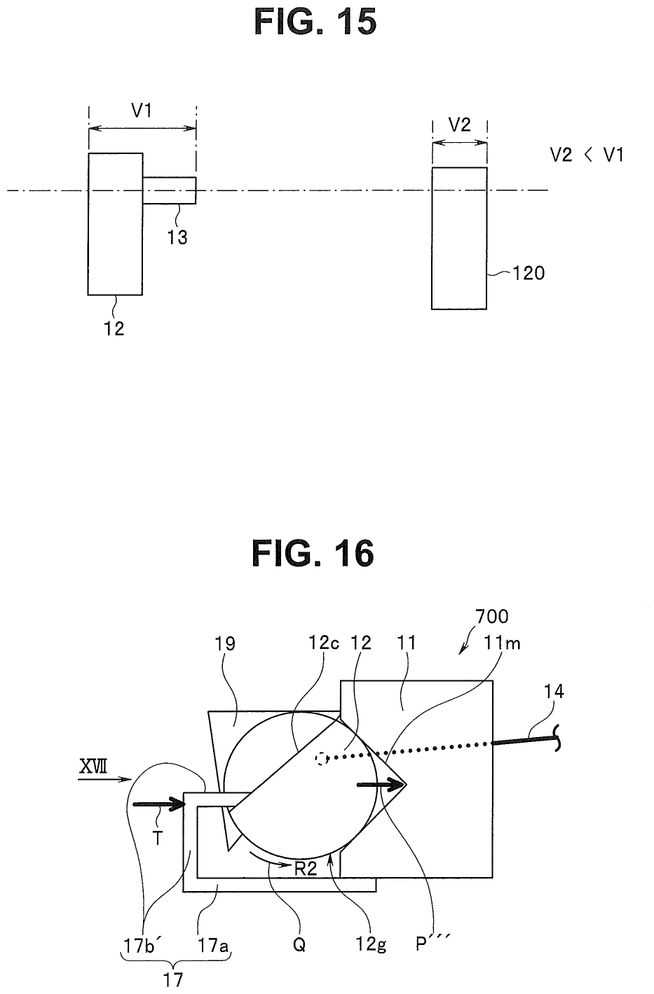

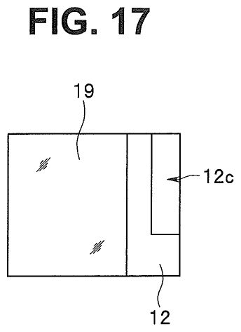

FIG. 16 is a side view illustrating a field of view direction variable mechanism provided in a distal end portion of an endoscope according to the present embodiment when a direction of a field of view is a front view direction, and FIG. 17 is a front view of a prism and a cam in FIG. 16 when seen in a direction XVII in FIG. 16.

Configurations of a distal end portion of an endoscope and an endoscope that includes the distal end portion of the endoscope according to a seventh embodiment is different in that the cam itself is fitted into a V-shaped bearing hole and a notch on which a pressing portion of an elastic member abuts is formed at the cam as compared with the distal end portion of the endoscope according to the aforementioned first embodiment illustrated in FIGS. 1 to 3, the distal end portion of the endoscope according to the fifth embodiment illustrated in FIG. 13, and the distal end portion of the endoscope according to the sixth embodiment illustrated in FIGS. 14 and 15.

Therefore, only the different points will be described, same reference numerals will be given to components that are similar to the components in the first, fifth, and sixth embodiments, and description of the components will be omitted.

As illustrated in FIG. 16, the embodiment has a configuration in which a cam 12 itself with a circularly formed outer circumferential surface 12g is fitted into the bearing hole 11m in a field of view direction variable mechanism 700.

As illustrated in FIGS. 16 and 17, a notch 12c on which a pressing portion 17b′ of an elastic member 17 abuts is formed at a part of the cam 12.

The pressing portion 17b′ presses the cam 12 against the bearing hole 11m with a force P″ and causes the second moment Q in the cam 12 by applying the elastic force T to the outer circumferential surface 12g of the cam 12.

Note that the other configurations are same as the configurations in the aforementioned first, fifth, and sixth embodiments.

With such a configuration, it is possible to reduce the scale of the field of view direction variable mechanism 700, that is, it is possible to realize reduction of the scale and the diameter of the distal end portion 2s in which the field of view direction variable mechanism 700 is provided similarly to the aforementioned sixth embodiment, and also, it is possible to more easily form the cam than in the sixth embodiment.

The other advantages are same as the advantages of the aforementioned first, fifth, and sixth embodiments.

Note that although the aforementioned first to seventh embodiments are described by exemplifying the field of view direction variable mechanism that causes the direction of the field of view of the endoscope to vary in the upward and downward directions, it is a matter of course that the present invention is not limited to the embodiments and can also be applied to a field of view direction variable mechanism of causing the direction of the field of view to vary in a left-right direction on the basis of the light receiving surface of the image pickup device.

Note that the present invention is not limited to the aforementioned embodiments and various modifications, changes, and the like can be made without changing the gist of the present invention.

1. A distal end portion of an endoscope comprising:
a first frame member at which a bearing is formed;
a second frame member to which an optical member is secured, the second frame member including a turning shaft turnably held by the bearing;
a cam disposed at the second frame member so as to be turnable around the turning shaft of the second frame member;
a traction member secured to the second frame member so as to apply, to the second frame member, a first moment of causing the second frame member to rotate in one direction through traction; and
an elastic member including a secured portion that is secured to the first frame member and a pressing portion that abuts on the cam while pressing the cam, the elastic member being configured to generate a second moment in a direction opposite to the direction of the first moment in the second frame member using a force applied to the second frame member through the pressing of the pressing portion to cause the second frame member to rotate in another direction.

2. The distal end portion of an endoscope according to claim 1,
wherein the cam includes a notch at a part of an outer circumferential surface with which the pressing portion comes into contact, and
turning of the cam is caused to stop by the pressing portion abutting on the notch due to the turning of the cam.

3. The distal end portion of an endoscope according to claim 1,
wherein the cam includes a notch at a part of an outer circumferential surface with which the pressing portion comes into contact, and
vibration is caused in the traction member by the pressing portion abutting on the notch due to turning of the cam.

4. The distal end portion of an endoscope according to claim 1,
wherein an outer circumferential surface of the cam with which the pressing portion comes into contact is formed to follow an involute curve based on the turning shaft.

5. The distal end portion of an endoscope according to claim 1,
wherein a circular or recessed-shaped bearing, into which the turning shaft is inserted, and by which the turning shaft is turnably held, is formed at the first frame member.

6. The distal end portion of an endoscope according to claim 1,
wherein the turning shaft configured to be substantially equal in thickness to a thickness of the cam and located so as to overlap with the thickness in plan view from a front side of the optical member is formed at a part of the cam, and
the turning shaft is turnably held by a recessed-shaped bearing formed at the first frame member.

7. An endoscope comprising:
the distal end portion of an endoscope according to claim 1.

8. The distal end portion of an endoscope according to claim 1, wherein the traction member is a traction wire.
